(19) United States
(12) Patent Application Publication
(10) Pub. No.: US 2006/0253103 A1
(43) Pub. Date: Nov. 9, 2006

(54) Removable cap needle access site

(76) Inventors: David S. Utterberg, Seattle, WA (US); William J. Schnell, Libertyville, IL (US)

Correspondence Address: Garrettson Ellis;Seyfarth Shaw LLP, Suite 4200, 55 East Monroe Street, Chicago, IL, 60603, US

(21) Appl. No.: 11/124,701

(22) Filed: May 9, 2005

(51) Int. Cl. A61M 39/10; B65D 41/00

(52) U.S. Cl. 604/533; 215/305; 215/321

(57) ABSTRACT

A one-piece elastomeric cap is proportioned to fit over an outer end of a female luer-lock connector. The cap comprises an outer cap member defining a generally cylindrical, bore-defining portion and an end wall to close off the bore. A generally cylindrical projection extends inwardly into the bore from the end wall. The projection is proportioned to sealingly fill at least an outer portion of the lumen of the female luer or luer-lock connector occupying said bore; the outer cap member preferably further defines at least one radially outwardly extending space adjacent to the end wall and extending radially outwardly from the bore, to receive outwardly-extending threads or lugs of the female luer-lock connector inserted within said cap.

### BACKGROUND OF THE INVENTION

In the field of administration and handling of medical solutions, blood, or the like, needleless connection sites are extensively used, particularly the well known male and female luer-lock connectors. These connectors generally comply with the appropriate ISO (International Organization for Standardization) specifications, so that on most medical sets and other devices they are very similar, and generally conform for interconnection with each other, a male luer connecting with a female luer. The term “luer-lock” implies that a locking connection is provided, particularly an extending, outer sleeve surrounding the male luer, which is threaded in the interior, and engages either outwardly projecting threads or lugs carried on the female luer-lock connector.

There is a problem, however, that can arise: needleless access sites for sets and other medical devices generally have no way to provide aseptic connection with a needle. There may be an important reason to provide medication or the like with a needle and syringe. Thus, an alternate, needle-penetrable access site generally has to be provided.

In accordance with this invention, a luer-type access site is provided, which also is capable of needle access, so that a single access site on medical equipment, such as an extracorporeal blood flow set or a parenteral solution administration set, is available for both connection with an injection needle, or connection with a male luer, as may be desired. Thus, by this invention, medical devices such as fluid flow sets may require fewer access sites, since one access site in accordance with this invention is capable of connecting with both a male luer and a hypodermic needle, resulting in convenience and cost saving.

Utterberg U.S. Pat. No. 5,071,413 discloses a connector which has a male luer taper shaped structure combined with a diaphragm-penetrating spike, for universal connection with both female luers and diaphragm connectors. By this invention, a female luer-based connector is provided for aseptic connection with a male luer or a hypodermic needle (or a blunt spike if desired).

### DESCRIPTION OF THE INVENTION

By this invention, a one-piece, needle pierceable, resealable, elastomeric cap is proportioned to fit over an outer end of the female luer-lock connector. The elastomeric cap comprises an outer cap member defining a tubular, bore-defining portion, and an end wall to close off the bore defined by said portion. A generally cylindrical projection extends inwardly into the bore from the end wall. This projection is preferably proportioned to sealingly fill at least an outer portion of the lumen of a female luer-lock connector (or luer connector) occupying the bore. The elastomeric cap preferably further defines at least one radially outwardly extending space adjacent to the end wall. This space extends radially outwardly from the bore, to receive outwardly-extending threads or lugs of a female luer-lock connector which is inserted within the cap.

The bore-defining portion, in some embodiments has an end opposed to the end wall which carries a thickened reinforcing portion, preferably an annular, thickened reinforcing portion to enhance inward pressure sealing of the bore-defining portion against a luer-lock connector, and to prevent inversion of the cap (contrary, for example, to the known, invertable Baxter I.V. bag closure). “Inversion” of the cap is a process as used in the Baxter I.V. bag closure to mount the closure on the bag, and involves the flipping of an outer sleeve from an original position where a tubular sleeve points away from the bag, then flipping by 180° to point toward the bag and to surround a tubular bag port, while a central, cylindrical projection occupies the bag port. Clearly, such a closure must be rather thin and highly flexible, and it is virtually impossible to manually mount it upon a port. Special machinery must be used.

To the contrary, in this invention, the one-piece elastomeric cap is more robust, so that it can typically be removed from, and also reapplied to, a female luer or luer-lock connector. As stated above, the thicker, one-piece elastomeric cap of this invention has a tubular, bore-defining portion that is preferably not invertible, and preferably carrying a thickened, reinforcing portion adjacent to the end opposed to the end wall, so that the cap can be manually removed, and then reapplied, if desired, while still remaining quite elastomeric, and particularly capable of needle penetration through the end wall and the generally cylindrical projection.

This elastomeric cap can provide sealing of a female luer-lock connector both inside of and outside of the tube of the female connector. The generally cylindrical projection can provide internal sealing of the lumen of the female connector, while the tubular, bore-defining portion of the outer cap member can provide external sealing against the female luer-lock connector.

The radially outwardly extending space defined in the outer cap member receives the outwardly extending threads or lugs of a female luer-lock connector, preferably in a manner which does not cause radial stretching of the elastomeric cap, which would otherwise take place if the threads or lugs of the female luer-lock connector were stretching side portions of the cap outwardly, which would also stretch the elastomeric cap at the end wall.

This can be disadvantageous. By this invention, the one piece, elastomeric cap serves both as a cap for a female luer, and as an access site for penetration by a hypodermic needle extending through the cap end wall, to gain access to the interior of the luer and to a container or conduit to which it is attached. If an area adjacent to the end wall is radially outwardly stretched, as by the threads of lugs of the female luer-lock connector, then the resealing capabilities of the elastomeric cap, after the needle is withdrawn, may be reduced. Accordingly, the radially outwardly extending space called for adjacent to the end wall, extending radially outwardly from the bore, may be provided to receive the outwardly extending threads or lugs of the female luer-lock connector, to avoid radial stretching resulting from the presence of the threads or lugs of the female luer-lock connector. Thus, needle resealablity of the system is increased.

Accordingly, the one-piece elastomeric cap of this invention also serves as a needle injection site, having an outer surface which is exposed and easily capable of sterile swabbing with an alcohol soaked swab. Alternatively, the cap may be removed, opening the female luer for access and connection with a male luer. Then, if desired, the cap can be manually replaced again on the female luer into its original position.

Preferably, the at least one radially outwardly extending space is a circular space, extending completely around the circumference of the luer, carried in the cap, in some embodiments without interruption.

Also, in some embodiments, the generally cylindrical projection has a length that exceeds the thickness of the remainder of the end wall from which it projects, so that it can occupy a substantial portion of the lumen of the female luer with which it connects.

The invention herein also relates to a female luer-lock connector which comprises a luer tube having a lumen, connected to a medical device for flow communication therewith, the luer-lock connector having an outer end. Connector threads or lugs are carried on the luer-lock connector adjacent to the outer end, for connection with a male luer-lock connector. The one-piece elastomeric cap described above may be attached, fitting over an outer end of the female luer-lock connector. The cap, as described above, has the generally cylindrical projection sealingly filling at least an outer portion of the lumen of the female luer-lock connector, and the radially outwardly extending space receives the outwardly extending threads or lugs of the female luer-lock connector.

Preferably, the cylindrical projection is proportioned to be of a diameter that it is generally under radial compression as it occupies the lumen of the luer-lock connector, to provide an inner area of annular compression and sealing. Also, in preferred embodiments, the tubular bore defining portion is proportioned to have a bore of a dimension so that it is held is a state of stretched, radial expansion by the female luer-lock connector, to provide an outer area of annular compression and sealing. Thus, two pressurized, sealed areas, inside and outside of the female luer, can be provided by the one-piece elastomeric cap of this invention.

The elastomeric cap may carry an outwardly projecting handle, if desired.

If it is desired to use the invention of this application with a female luer connector which is not a luer lock connector, and thus has no outwardly projecting screw threads or lugs, the outer cap member does not need to define the radially outwardly extending space adjacent to the end wall, while still providing the other desired advantages of this invention, such as sealing of the luer tube inside and out, while providing a needle penetrable injection site, coupled with a removable sealing cap, which can be taken away to expose the female luer for connection with a male luer.

### DESCRIPTION OF THE DRAWINGS

In the drawings, FIG. 1 is an exploded, elevational view of a one-piece elastomeric cap in accordance with this invention, and a female luer-lock member to which the cap can attach.

FIG. 2 is a sectional view taken along line 2-2 of FIG. 1.

FIG. 3 is a perspective view of the one-piece elastomeric cap and female luer-lock connector of FIG. 1.

FIG. 4 is an enlarged, vertical sectional view of the one-piece elastomeric cap and female luer-lock connector similar to the previous drawings, shown in their connected relationship.

FIG. 5 is an elevational view of another embodiment of a one-piece elastomeric cap, shown connected on a female luer-lock connector.

FIG. 6 is a vertical, sectional view taken along line 6-6 of FIG. 5.

FIG. 7 is a perspective view of FIG. 5.

### DESCRIPTION OF SPECIFIC EMBODIMENTS

Referring to FIGS. 1-4, a one-piece, molded elastomeric cap 10 is provided, made of polyisoprene, for example, and being proportioned to fit over an outer end 12 of a female luer-lock connector 14, which may be of conventional design. Female luer-lock connector 14 may, in turn, be heat sealed or otherwise secured to a fitting on a medical device 30 at its inner end 16, such as a fitting mounted on a tubular blood or solution flow set, such as an extracorporeal blood set for hemodialysis, pheresis, hemoperfusion, or the like, or a parenteral solution set. Or, female luer-lock connector 14 may be mounted on a syringe 30, a solid casing of a medical device, or the like.

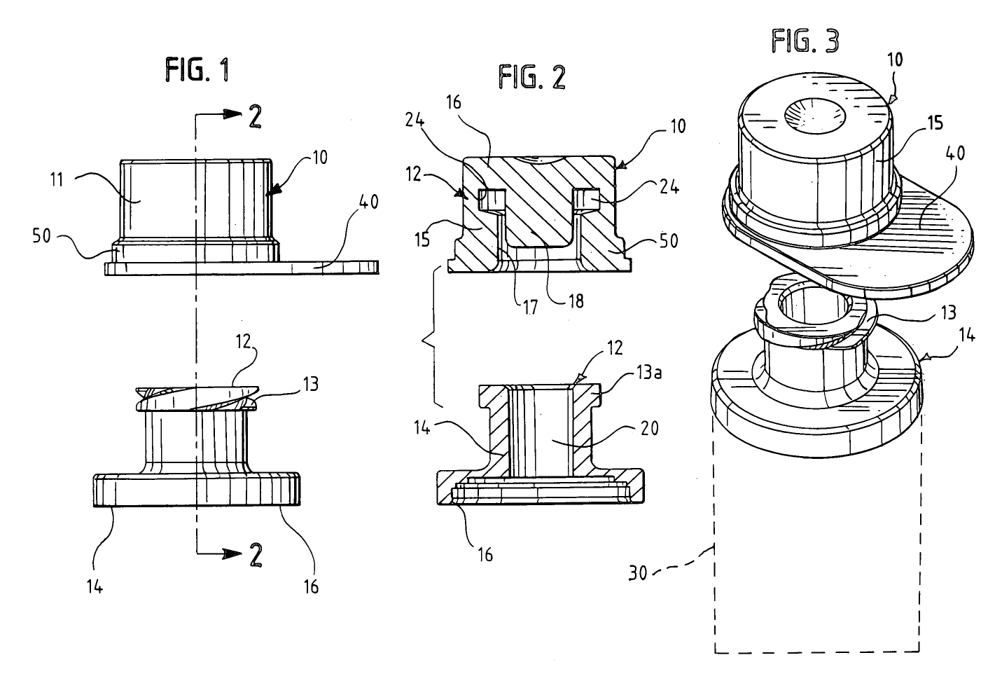

Cap 10 may comprise a one-piece, molded, elastomeric member utilizing conventional, known, medically suitable elastomeric materials. Cap 10 comprises an outer cap member 12, which further defines a tubular bore 17, defined by a corresponding bore-defining portion 26 of outer cap member 12 as can be seen in FIG. 2. Also, an end wall 16 closes off bore 17.

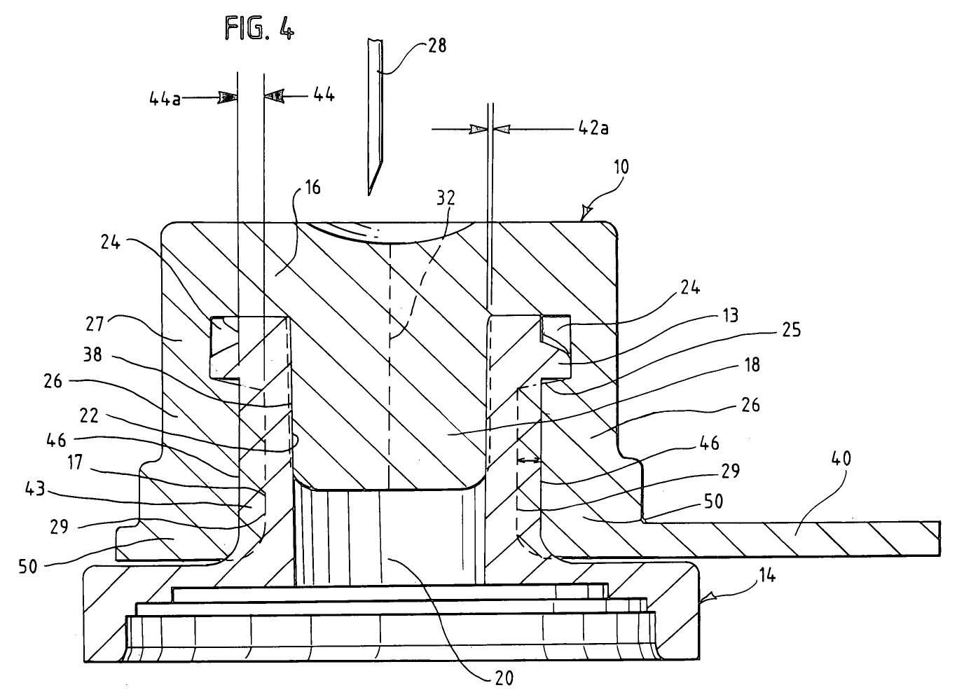

Cap 10 also defines a generally cylindrical projection 18, which extends inwardly into the bore 17 from end wall 16. Projection 18 is proportioned to sealingly fill at least an outer portion 22 of the lumen 20 of a female luer-lock connector 14, as can be seen from FIG. 4.

Outer cap member 12 further defines a circular, radially outwardly extending space 24, which is adjacent to end wall 16 and extends radially outwardly from bore 17, to receive the outwardly extending threads or lugs 13, 13a of female luer-lock connector 14. As previously stated, FIGS. 1 and 3 show such threads 13, while FIG. 2 has been modified to show a pair of diametrically opposed lugs 13a, both of which structures are conventional and known. Because of the presence of annular, outwardly extending space 24 adjacent to end wall 16, the area 27 of cap 10 (FIG. 4) is not stretched outwardly by the presence of screw threads or lugs 13. Thus, the material of end wall 16 is thus not stretched outwardly either, so that a needle 28, connected to a syringe or the like, can penetrate through end wall 16 and cylindrical projection 18 into an open portion of lumen 20, for access to a medical device 30 (FIG. 3) to which luer-lock connector 14 is secured. Then, needle 28 can be withdrawn and, because of the absence of stretching in the volume of end wall 16, resealability is better maintained despite the presence of the outwardly extending threads or lugs 13.

Area 27 is preferably at least about 0.8 to one mm. thick, being the thinnest portion of outer cap member 12.

If desired, end wall 16 and cylindrical projection 18 may carry a slit 32, so that there may be penetration by a blunt needle, slit 32 being resealable upon withdrawal of the needle.

It can be seen that cylindrical projection 18 has a length, projecting below end wall 16, which projecting length exceeds the thickness of end wall 16, to increase internal sealing at the cylindrical internal sealing area 38. Also, optionally, one-piece, elastomeric cap 10 may carry an outwardly projecting handle 40, to facilitate removal of the cap from luer-lock connector 14.

The double line 42, 42a is an indication that generally cylindrical portion 18 has an outer diameter that is slightly larger, for example by about 0.05 mm., than the inner diameter of lumen 20 of luer-lock connector 14. The cylindrical tube 43 of connector 14 is generally rigid, while projection 18 is made of elastomer, so that projection 18 is compressed to a degree, to define the previously mentioned, inner area of annular compression and sealing 38. FIG. 4 is substantially a drawing to scale, showing exemplary, effective material thicknesses for the cap of this invention.

Double line 44, 44a illustrates the expansion which is provided by the outer diameter cylindrical surface 46 of luer-lock connector 14, which stretches tubular, bore defining portion 26 outwardly from its normal, as-molded configuration 29, to resiliently press against cylindrical luer surface 46, and thus to provide the above described outer area of annular compression and sealing. Surface 29 thus has been driven outwardly to squeeze the area of surface 46 when cap 10 is placed upon female luer-lock connector 14. The horizontal distance between lines 44 and 44a may be 0.55 mm., by way of example.

Thus, an inner and an outer area of annular compression and sealing is provided around the tubular projection of luer-lock connector 14, providing effective, microorganism blocking sealing of the luer-lock connector.

As previously described, needle 28 may penetrate through the elastomeric cap 10, or, alternatively, cap 10 may be removed from luer-lock connector 14 by manual grasping of handle 40 and removal of the cap, for access to connector 14. If desired, cap 10 then may be replaced to reclose luer-lock connector 14 at any time.

The bore-defining portion 26 of cap 10 is of generally tubular shape, and has an end opposed to end wall 16 which carries an annular, thickened reinforcing portion 50, which enhances the desirable, inward pressure sealing of bore-defining portion 26 against luer-lock connector 14, to provide an effective, outer area 46 of annular compression and sealing. Because of the presence of the thickened, reinforcing portion 50, inversion of cap 10 is not practicable. Instead, cap 10 has sufficient body and thickness of dimension that it may be easily and manually placed again on the luer-lock connector 14 after it has been removed, for a reclosing thereof.

The presence of threads 13 or lugs 13a on luer-lock connector 14 provides to the cap emplaced on the luer lock connector a good resistance to blow off during sterilization or other conditions of increased internal pressure, so that elastomer cap 10 reliably provides a seal, until it is positively removed manually. Preferably, radially outwardly extending space 24, which is typically a complete, annular space extending 360°, has a lower edge 25 which is at least nearly perpendicular to the axis of the cap 10, similar in direction to line 32, to firmly retain threads or lugs 13, 13a so that cap 10 cannot be popped off when high pressures are present in lumen 20, and in the medical device to which luer connector 14 is attached. Typically this angle to the axis is no more than about 20° away from perpendicular to the axis of the cap 10.

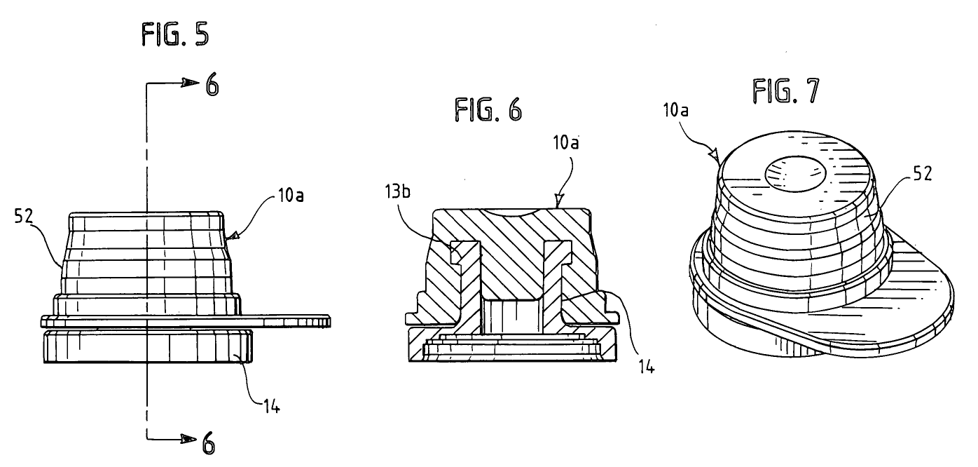

Turning to FIGS. 5 through 7, another embodiment 10a of the cap of this invention is disclosed, shown to be carried on a conventional female luer-lock connector 14 of a design similar to the connector of the previous embodiment, and having diametrically outwardly projecting lugs 13b of conventional design rather than threads. This embodiment is similar to the previous embodiment, except as otherwise described herein. Specifically, the outer, vertical surface 52 of cap 10a tapers inwardly as it extends upwardly to a certain extent. Otherwise, the components are similar in structure and in function.

Thus, a cap is provided which has sufficient bulk and rigidity, while being basically elastomeric, to permit its being pushed onto a female luer-lock connector over the threads or lugs thereof without collapse or the need for inversion of the bore-defining portion, rather as a rigid cap might be inserted onto the luer lock connector. The cap of this invention can maintain and resist internal pressure. This is accomplished with a cap which is made of a single, molded, elastomeric piece, rather than being of multiple piece construction, which of course represents a manufacturing advantage that can be reflected in a lower cost.

The above has been offered for illustrative purposes only, and is not intended to limit the scope of the invention of this application, which is as defined by the claims below.

That which is claimed is:

1. A one-piece, elastomeric cap proportioned to fit over an outer end of a female luer-lock connector, which comprises:
an outer cap member defining a tubular, bore-defining portion, and an end wall to close off the bore of said portion;
a generally cylindrical projection extending inwardly into said bore from the end wall, said projection being proportioned to sealingly fill at least an outer portion of the lumen of a female luer-lock connector occupying said bore;
said outer cap member further defining at least one radially outwardly extending space adjacent to said end wall, said space extending radially outwardly from said bore, to receive outwardly-extending threads or lugs of a female luer-lock connector inserted within said cap.

2. The elastomeric cap of claim 1 in which said bore-defining portion is of generally tubular shape, having an end opposed to said end wall which carries an annular, thickened, reinforcing portion to enhance inward pressure sealing of the bore-defining portion against said luer-lock connector and to prevent inversion of the cap.

3. The elastomeric cap of claim 1 which carries an outwardly projecting handle.

4. The elastomeric cap of claim 1 in which said at least one radially outwardly extending space extends completely around the circumference of said bore.

5. The elastomeric cap of claim 1 in which the end wall and cylindrical portion are proportioned to be needle pierceable, and resealable when the needle is withdrawn.

6. The elastomeric cap of claim 1 in which the generally cylindrical projection has a length that exceeds the thickness of the remainder of the end wall.

7. The elastomeric cap of claim 1 in which said bore-defining portion is of generally tubular shape, having an end opposed to said end wall which carries an annular, thickened, reinforcing portion to enhance inward pressure sealing of the bore-defining portion against said luer-lock connector and to prevent inversion of the cap, and in which said at least one radially outwardly extending space extends completely around the circumference of said bore.

8. The elastomeric cap of claim 7 which carries an outwardly projecting handle.

9. A female luer-lock connector which comprises a luer tube having a lumen, said luer lock connector being connectable at an inner end to a medical device for flow communication therewith, said luer-lock connector having an outer end, connector threads or lugs carried on said luer lock connector adjacent to said outer end for connection with a male luer-lock connector, in combination with:
a one-piece, elastomeric cap fitting over the outer end of said female luer-lock connector, said cap comprising:
an outer cap member defining a tubular bore-defining portion receiving said female luer lock connector in the bore, and an end wall to close off the bore of said portion and the outer end of said female luer lock connector;
a generally cylindrical projection extending inwardly into said bore and the female luer lock connector from the end wall, said projection sealingly filling at least an outer portion of the lumen of said female luer-lock connector;
said outer cap member further defining at least one radially outwardly extending space adjacent to said end wall and extending radially outwardly from said bore, said space receiving the outwardly extending threads or lugs of said female luer-lock connector.

10. The female luer-lock connector and cap of claim 9 in which said generally cylindrical projection is under radial compression, to provide an inner area of annular compression and sealing.

11. The female luer-lock connector and cap of claim 9 in which said bore-defining portion is held in a state of stretched, radial expansion by the female luer-lock connector, to provide an outer area of annular compression and sealing.

12. The female luer-lock connector and cap of claim 11 in which said tubular bore-defining portion has an end opposed to said end wall which carries an annular, thickened, reinforcing portion to enhance inward compression sealing of the bore-defining portion against said luer-lock connector and to prevent inversion of the cap.

13. The female luer-lock connector and cap of claim 12 in which said generally cylindrical projection is under radial compression, to provide an inner area of annular compression and sealing.

14. The female luer-lock connector and cap of claim 11 in which said generally cylindrical projection is under radial compression, to provide an inner area of annular compression and sealing.

15. The female luer-lock connector of claim 9 in which the connector is connected to a medical device at said inner end.

16. The female luer-lock connector and cap of claim 9 in which said at least one radially outwardly extending space extends completely around the circumference of said bore.

17. The female luer-lock connector and cap of claim 9 in which the cap is proportioned to be needle pierceable, and resealable when the needle is withdrawn.

18. The female luer-lock connector and cap of claim 9 in which the generally cylindrical projection has a length that exceeds the thickness of the remainder of the end wall.

19. A one-piece, elastomeric cap proportioned to fit over an outer end of a female luer connector, said cap comprising a tubular, bore-defining portion, and an end wall to close off the bore of said portion;
a generally cylindrical projection extending inwardly into said bore from the end wall, said projection being proportioned to sealingly fill at least an outer portion of the lumen of a female luer connector occupying said bore, the tubular bore-defining portion having an end opposed to said end wall which carries an annular, thickened, reinforcing portion, to enhance inward pressure sealing of the bore-defining portion against said luer connector and to prevent inversion of the cap.

20. The one-piece, elastomeric cap of claim 19 in which the end wall and cylindrical portion are proportioned to be needle pierceable, and resealable when the needle is withdrawn.

21. The one-piece elastomeric cap of claim 19 in which the generally cylindrical projection has a length that exceeds the thickness of the remainder of the end wall.

22. A kit which incorporates a medical device carrying a female luer-lock connector, and the one-piece, elastomeric cap of claim 19.

23. A kit which incorporates a medical device carrying a female luer-lock connector, and the one-piece, elastomeric cap of claim 1.
